(19) United States
(12) Patent Application Publication
(10) Pub. No.: US 2008/0194980 A1
(43) Pub. Date: Aug. 14, 2008

(54) Method of and Unit for Determining the Cardiac Output of the Human Heart

(75) Inventors: Janneke Gisolf, Haarlem (NL); Gijs Steenvoorden, Pijnacket (NL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: NEDERLANDSE ORGANISATIE VOOR TOEGEPASTNATUURWETENSCHAPPELIJK ONDERZOEK, Delft (NL)

(21) Appl. No.: 11/718,696

(22) PCT Filed: Nov. 5, 2004
(86) PCT No.: PCT/NL2004/000783

§ 371 Date: Feb. 15, 2008

(51) Int. Cl. A61B 5/029; A61M 16/00

(52) U.S. Cl. 600/526; 128/204.18

(57) ABSTRACT

A method of determining the cardiac output of a human heart, comprising: providing a sensor for measuring an expiratory tidal volume; a sensor for measuring an oxygen uptake from the expiratory tidal volume; a sensor for measuring a CO2 partial pressure in the expiratory tidal volume; and providing a circulation model for relating a measured CO2 partial pressure and oxygen uptake to a heart stroke volume per breath. The measured expiratory tidal volume, oxygen uptake and CO2 partial pressure are inputted in said circulation model and a heart stroke volume per breath consistent with said circulation model is calculated.

Today, a number of techniques is available for human cardiac output monitoring. They can be based on invasive or non-invasive measurement techniques and are developed for continuous or non-continuous monitoring purposes. One continuous monitoring method is described as “Modelflow” in U.S. Pat. No. 5,400,793. This method couples the pulse-type blood-stream pressure signal derived from the aorta and calculates the flow where the aorta is regarded as a transmission line supplemented by a windkessel compliance.

Cardiac output monitoring, for instance, of patients who undergo major surgery, or are at intensive care, provides valuable information on patient status. Non-continuous methods are reliable, but are often invasive and require considerable skill in conducting the measurement. Continuous cardiac output monitoring is therefore becoming more and more popular. Therefore, methods and techniques for continuous and non-invasive cardiac output monitoring are important for future developments in the field of cardiac output monitoring.

For non-invasive situations, the above mentioned “Modelflow” technique requires the availability of a non-invasive blood pressure signal which is not always available at the operation- or intensive care units. Furthermore, the Modelflow technique must be calibrated using other complex cardiac output measuring techniques. The invention aims at providing a reliable continuous measurement of cardiac output, at the same time obviating a necessity for performing complex or invasive measurements or calibration. The invention aims at providing a reliable monitor of cardiac output, both for output changes and absolute output measurements.

To this end, the invention provides a method according to the features of claim 1. Specifically, by providing a circulation model wherein a measured CO2 partial pressure and oxygen uptake to a heart stroke volume per breath are related to the cardiac output, minimal attributes are necessary for providing a reliable measurement. In another aspect, the invention provides a ventilation unit for actively or passively ventilating the lungs and comprising a breathing mask piece according to claim 22. In particular, the invention provides a ventilation unit comprising:

a respiratory detector for measuring an expiratory tidal volume and respiratory rate;

an oxygen sensor for measuring an oxygen uptake from the expiratory tidal volume;

a CO2 sensor for measuring a CO2 partial pressure in the expiratory tidal volume; and

a processor programmed in consistency with a circulation model, for relating a measured CO2 partial pressure, expiratory tidal volume and oxygen uptake to a heart stroke volume per breath;

wherein the respiratory detector, the oxygen sensor and the CO2 sensor are arranged to be coupled to said processor for inputting a measured expiratory tidal volume; a measured oxygen uptake VO2 and a measured CO2 partial pressure, and the processor arranged to output a heart stroke volume per breath to an output unit consistent with said circulation model.

The inventors have found that variations of the CO2 partial pressures can be modelled using a mathematical model of the circulation system. Accordingly, by the invention, using capnography and a breath-to breath model, the cardiac output of the heart can be monitored non-invasively in a continuous manner.

In a preferred embodiment, said circulation model defines a distribution of apical and basal lung segments, each segment defining a predetermined ventilation perfusion ratio (V/Q), and where the heart stroke volume per breath n SVₙ is calculated to be consistent with an estimated fraction b of CO2 in air in each segment, derived from a measured end tidal CO2 partial pressure and pulmonary oxygen uptake. The ventilation and perfusion rates that the model takes into consideration may incorporate physical or pathological conditions of the circulation in a subject that is monitored.

On standing up, the distribution of blood flow over the lung changes due to hydrostatic pressure differences from apex to base in the upright position. Distribution of tidal volume also changes, due to an altered pressure/volume relationship of the lung compartments. Consequently, the overall ventilation/perfusion ratios changes. Results indicate that the model accurately estimates end tidal variations of the CO2 partial pressures during posture change and tracks spontaneous variations in partial CO2 pressure (P_{CO2}) in a fixed body position, substantially ascribed to changes in cardiac output. It is noted that parts of this model were disclosed in a scientific paper of the inventor: Janneke Gisolf et al, “Tidal Volume, Cardiac output and functional residual capacity determine end-tidal CO2 transient during standing up in humans”, J Physiol. 554.2 p 579-590.

Further features and benefits will become apparent from the description read in conjunction with the figures.

In the figures:

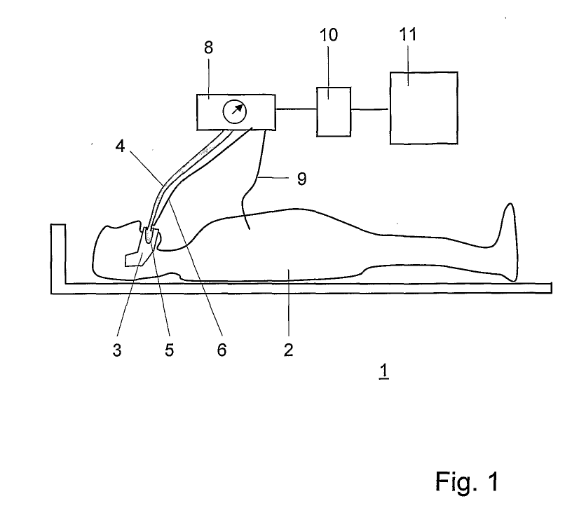

FIG. 1 illustrates a schematic illustration of a ventilation unit according to the invention;

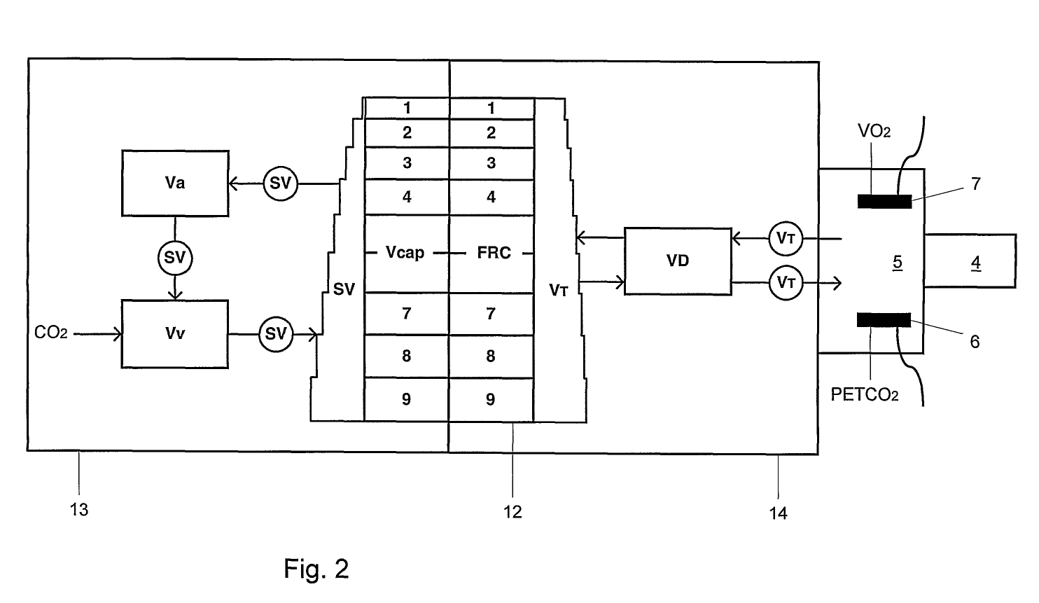

FIG. 2 illustrates a schematic illustration of an embodiment a circulation model for relating a measured CO2 partial pressure and oxygen uptake to a heart stroke volume per breath; and

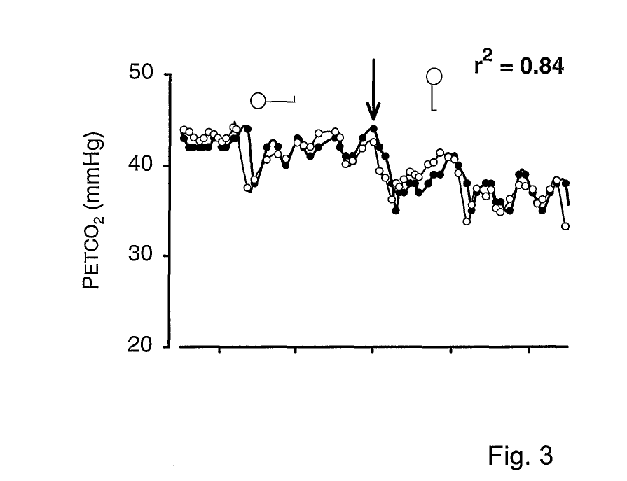

FIG. 3 illustrates an individual end tidal CO2 partial pressures (PETCO2) recordings and a model simulations during lying down and standing in a test situation.

In FIG. 1 a schematic drawing is illustrated of an intensive care unit 1 where a person 2 is actively or passively ventilated, for instance, during surgery etc. To this end a ventilation mask 3 is connected to the mouth and breathing air is supplied via a duct 4 for ventilating the lungs. In or near the mouthpiece 5 of the duct 4 a sensor 6 is provided for measuring an end-tidal CO2 partial pressure in the expiratory tidal volume. An oxygen uptake sensor 7 is shown near the mouthpiece but may also be provided more distant in an automatic ventilating machine 8. Furthermore, a detector 9 is present for detecting a respiratory rate. In the ventilating machine 8 furthermore detectors are present for measuring a breathing tidal volume, in particular, an expiratory tidal volume. The measured expiratory tidal volume, oxygen uptake and a measured end-tidal CO2 partial pressure are fed into the processor 10. In addition to an instantaneous end-tidal CO2 partial pressure, also, an time-integrated CO2 partial pressure per breath can be measured and fed into the processor 10.

The processor 10 is programmed in consistency with a circulation model which defines a distribution of apical and basal lung segments, each segment defining a predetermined ventilation perfusion ratio (V/Q), and where the heart stroke volume per breath n is calculated to be consistent with an estimated fraction of CO2 in air in each segment, derived from the measured end tidal CO2 partial pressure and pulmonary oxygen uptake.

The ventilation and perfusion rates that the model takes into consideration may incorporate physical or pathological conditions of the circulation in a subject that is monitored. The model is further detailed in FIG. 2. From the processor 10, an output value, representing a measure for cardiac output is outputted to a monitoring device 11, for instance, a screen or a processing arrangement that is active in the intensive care monitoring.

As illustrated in FIG. 2, in a special embodiment, the circulation model defines a distribution of apical and basal lung segments 12, each segment, from top to basal lung segment, contributing to a constant ventilation/perfusion ratio V/Q in supine position or a decreasing ventilation/perfusion ratio V/Q in a standing position. In the model, the heart stroke volume per breath n SVn is calculated to be consistent with an estimated fraction b of CO2 in air in each segment, derived from a measured end tidal CO2 partial pressure an pulmonary oxygen uptake and further exemplified in Equation 16 of the model equations defined further below.

As illustrated in FIG. 2, the circulation model defines a circulated total blood volume 13 and a ventilated total air volume 14. To assess the cardiac output, a summed CO2 content in said total blood/air volume is calculated as will be further illustrated below. In the illustrated embodiment the model contains 9 lung segments 12. The model further defines a venous compartment Vv; an arterial compartment Va; and a fixed blood volume of segmented lung capillaries Vcap; and a variable heart stroke volume per breath SVn distributed over the segments; and wherein said ventilated total air volume comprises a fixed volume of a segmented functional residual capacity FRC; a variable expiratory tidal volume VTn distributed over the segments; and an anatomical dead space VD. Each segment's share of the FRC and VT is determined by its position with the apical segments smaller than the basal segments. Using an established relation between anatomical VD and height (Hart et al. 1963), the model VD can be set for men at a greater volume compared to the VD for women (1.4 times), for instance, with the VD at 200 ml for men and 140 ml for women in the supine position. In the upright position, these values may be increased by 70 ml. Alternatively, the anatomical dead space can be measured using known measurement techniques.

The respiratory quotient (RQ), defined as the ratio of carbon dioxide production (VCO2) to VO2, normally between 0.7 and 1.0, was set fixed at 0.9. Alternatively, variable values of the RQ value can be inputted in the model using known measurement techniques. The consequences of variations in oxygen uptake are 2-fold:

1. oxygen uptake is related to basal metabolism, and is related to CO2 production. For a resting, supine measurement there will be little variation in oxygen uptake.

2. the level of oxygenation of blood determines its ability to carry CO2 (known as the ‘Haldane effect’. For a normal, resting measurement the oxygenation will be optimal and the CO2 uptake will be defined by the Equation 1 detailed below.

The lung capillary volume and the small venule volume are lumped together, as gas exchange occurs in both. The major arteries of the lung are included in the venous compartment; the major veins of the lung are included in the arterial compartment. In a practical example, the total blood volume of 5.5 l is distributed over Vv (4.0 l), Va (1.3 l) and Vcap (0.2 l). The segmented model may include the effects of gravity to gravity-induced blood perfusion gradient in the lung. In the supine position, SV and VT are distributed equally over all compartments. With nine compartments, in the supine position each lung compartment receives one-ninth of the breath-to-breath SV and VT. In the upright position there is an apical-to-basal perfusion and ventilation gradient, with increased perfusion and ventilation at the lung base. The perfusion gradient is steeper than the ventilation gradient, resulting in a 7.9-0.8 apical-to-basal V/Q gradient. Furthermore, different values for anatomical dead space VD may be used going from supine to upright respiratory positions, for instance in a range varying from +53 ml (anatomical) to +81 ml (physiological).

Table 1 defines a distribution of stroke volume (SV), tidal volume (VT), functional residual capacity (FRC) and lung capillary blood volume (Vcap) per lung segment k, in the supine and standing position, that can be included in the model. Upright distributions are based on measurements previously performed by West J B (1962): “Regional differences in gas exchange in the lung of erect man”. J Appl Physiol 17, 893-898 West (1962). For the upright position, the FRC is increased with respect to the supine position.

TABLE 1

| Lung compartment | | | | | | | | | | |
|---|---|---|---|---|---|---|---|---|---|---|
| (apical to basal, | | | | | | | | | | |
| respectively) | | 1 | 2 | 3 | 4 | 5 | 6 | 7 | 8 | 9 |
| Supine | | | | | | | | | | |
| Perfusion | (% SVk) | 11.1 | 11.1 | 11.1 | 11.1 | 11.1 | 11.1 | 11.1 | 11.1 | 11.1 |
| (Q) | | | | | | | | | | |
| Ventilation | (% VTk) | 11.1 | 11.1 | 11.1 | 11.1 | 11.1 | 11.1 | 11.1 | 11.1 | 11.1 |
| (V) | | | | | | | | | | |
| Alveolar | (% FRCk) | 6.58 | 8.64 | 10.11 | 11.16 | 11.90 | 12.43 | 12.81 | 13.08 | 13.27 |
| vol. | | | | | | | | | | |
| Lung capil. | (% Vcapk) | 6.58 | 8.64 | 10.11 | 11.16 | 11.90 | 12.43 | 12.81 | 13.08 | 13.27 |
| Standing | | | | | | | | | | |
| Perfusion | (% SVk) | 0.58 | 3.21 | 5.84 | 8.47 | 11.10 | 13.73 | 16.36 | 18.99 | 21.62 |
| (Q) | | | | | | | | | | |
| Ventilation | (% VTk) | 4.58 | 6.63 | 8.48 | 10.13 | 11.62 | 12.96 | 14.17 | 15.25 | 16.22 |
| (V) | | | | | | | | | | |
| Alveolar | (% FRCk) | 6.58 | 8.64 | 10.11 | 11.16 | 11.90 | 12.43 | 12.81 | 13.08 | 13.27 |
| vol. | | | | | | | | | | |
| Lung capil. | (% Vcapk) | 6.58 | 8.64 | 10.11 | 11.16 | 11.90 | 12.43 | 12.81 | 13.08 | 13.27 |

For the purpose of tracking short-term end tidal P_{CO2} variations with posture change, data was selected starting 150 s prior to standing up and ending 150 s after standing up. Mean arterial blood pressure was measured with a Finapres (Model 5; Netherlands Organization for Applied Scientific Research, Biomedical Instrumentation, TNOBMI). The cuff was applied to the midphalanx of the middle finger of the dominant arm, which was placed at heart level. Beat-to-beat changes in SV were estimated by modeling flow from arterial pressure (Modelflow, TNOBMI). This method computes an aortic waveform from a peripheral arterial pressure signal using a non-linear 3-element model of the aortic impedance (Jellema et al. 1999; Harms et al. 1999). Cardiac output was the product of SV and HR. To obtain absolute values of Q to calibrate Modelflow Q, a Fick-determined Q was obtained from arterial and central venous O2 content and the VO2 in the supine and in the standing position. Absolute values of Q were used to calibrate Modelflow Q, averaged during 150 s in the supine position, and during 150 s of standing. Breath-to-breath online gas analysis was performed using a Medical-Graphics CPX/D metabolic cart. Respiratory gas was sampled continuously from a mouthpiece and partial gas pressures were obtained from a Zirkonia oxygen analyser (accuracy±0.03% O2) and a non-dispersive infrared sensor for CO2 (accuracy±0.05% CO2) that thus delivered VO2, VCO2and PETCO2.

FIG. 3 illustrates a measured sample where breath-to-breath end tidal partial CO2 pressures are recorded for an individual subject, to verify the validity of the circulation model. The figure contains a plot of breath-to-breath PETCO2 measurements (·) during 150 s supine and 150 s of standing, and a model simulation (O) of the same period. Arrows indicate posture change from supine to standing at time zero. Inputs to the model were (measured) breath-to-breath values of VT, SV (summed per breath) and VO2. Starting values for PCO2 in the venous and the arterial blood and in the various lung compartments were set for each test subject, corresponding to their starting measured PETCO2. Venous CO2 concentrations were set at a starting value ranging from 52 to 55%. The PCO2 starting values in arterial blood and the lung compartments ranged from 40 to 42 mmHg. The model tracks PETCO2 during standing up, and it also follows non-posture-related variations in PETCO2 (r²=0.43-0.86), with those registrations with the greatest variance in measured PETCO2 resulting in the best correlations of MPETCO2 with PETCO2 (P<0.01).

### Model Equations

For the definitions of the various quantities used in the model equations reference is made to Table 2.

TABLE 2

| Symbols | Definition | Units |
|---|---|---|
| [CO2]a | Arterial CO2 content | % |
| [CO2]v | Venous partial CO2 content | % |
| ABP | Arterial blood pressure | mmHg |
| FRC | Functional residual capacity | ml |
| HR | Heart rate beats | min-1 |
| PETCO2 | End-tidal partial CO2 pressure | mmHg |
| PkCO2 | Lung compartment k partial CO2 pressure | mmHg |
| Q | Cardiac output | l min-1 |
| RQ | Respiratory quotient | unitless |
| R-R | Respiratory rate | min-1 |
| SV | Stroke volume per breath | ml |
| TRESP | Respiratory interval | s |
| Va | Arterial blood volume | ml |
| Vcap | Lung capillary blood volume | ml |
| VD | Anatomical dead space | ml |
| VE | Ventilation | l min-1 |
| V O2 | Pulmonary O2 uptake | ml min-1 |
| V/Q | Ventilation/perfusion ratio | unitless |
| VT | Tidal volume | ml |
| Vv | Venous blood volume | ml |

### Conversion and Weight Functions.

The CO2 equilibrium curve relating blood CO2 content ([CO2]) to blood partial CO2 pressure (PCO2) is described as [CO2]=f (PCO2),with

f(x)=0.53(1.266−exp(−0.0257x)) Equation 1

To compute PCO2 from [CO2] in blood, we use the inverse function

f−1(x)=−ln(1.266−(x/0.53))/0.0257 Equation 2

To convert PCO2 in air (mmHg) to [CO2] (%), we use the conversion factor c, which amounts to 0.1316% mmHg−1. The distribution of SV and VT over each lung compartment k (k=1 . . . 9) is described by functions g and h, respectively. These functions, which are different for the supine and upright positions and yield the fractions for SV and VT listed in Table 2, are given by

g(k)= 1/9(in the supine position)−0.0205+0.0263k (in the upright position) Equation 3

and

h(l)= 1/9 (in the supine position) 0.226(1.102−exp(−0.1063k) (in the upright position)

Each lung compartment's share of FRC, Vcap and VD is given by the weight function

w(k)=0.10055(1.36708−exp(−0.3393k)) Equation 5

which yields the fractions for FRC and Vcap listed in Table 2.

### Venous CO2.

For each breath n, a variation in CO2 in said venous compartment Vv is expressed by the amount A that arrives from the arterial compartment Va plus the amount B of CO2 created by the basal metabolism minus the amount C that exits the venous compartment; where the sum of CO2 created by the basal metabolism is expressed as a function of the oxygen uptake VO2 per breath. For each breath a, the venous CO2 content ([CO2]v,n) is calculated from its previous value [CO2]v,n−1 according to Equations 6-9. The amount of CO2 in the venous compartment increases by the amount that arrives from the arterial compartment (A) and the amount created by the basal metabolism (B), and decreases by the amount that leaves the compartment (C). Thus, we have

[CO2]v,n−[CO2]v,n−1+(A+B−C)/Vv

where

C=[CO2]v,n−1SVn Equation 7

A=[CO2]a,n−1SVn Equation 8

with [CO2]a denoting the arterial CO2 content, and

B=VO2, nRQ(TRESP,n/60) Equation 9

where VO2,n is the oxygen extraction for breath n (in ml min−1) and RQ is the respiratory quotient, which is set at 0.9 (the average as approximated from subject data, by dividing VCO2by VO2). The term is multiplied by the breath duration (in min) (TRESP,n/60) to estimate the CO2 produced per breath.

### Arterial CO2.

For each breath n, a variation in CO2 in said venous compartment Vv is expressed by the amount A that arrives from the arterial compartment Va plus the amount B of CO2 created by the basal metabolism minus the amount C that exits the venous compartment; where the sum of CO2 created by the basal metabolism is expressed as a function of the oxygen uptake VO2 per breath.

The arterial blood CO2 content for breath n ([CO2]a,n) is calculated from its previous value [CO2]a,n−1 according to Equations 10-12. The amount of CO2 in the arterial compartment increases by the amount of CO2 arriving from the lungs (D) and decreases by the amount of CO2 leaving the arterial compartment (E)

[CO2]a,n=[CO2]a,n−1+(D−E)/Va Equation 10

The amount D can be estimated from the end-tidal partial CO2 pressure in each lung compartment k (PkCO2k=1 . . . 9) through

$$D = {\sum }_{k = 1}^{9} f ( PkCO 2 , n - 1 ) ( g ( k ) SVn ) \qquad Equation 11$$

Where f is the above function that relates blood CO2 content to the blood partial CO2 pressure and g is the above function that defines the distribution of SV over the nine lung compartments. The amount E is given by

E=[CO2]a,n−1SVn Equation 12

### Lung CO2.

For each breath n, a CO2 amount in each segment k of said segmented lung model is expressed as the amount F of CO2 in the lung capillaries Vcap, in the functional residual capacity FRC, and in the anatomical dead space VD, as a function of an estimated CO2 partial pressure PₖCO2ₙ in the segments k; plus the amount G of CO2 carried to the lungs from the venous compartment by the heart stroke volume SVₙ; and where the estimated CO2 partial pressure PkCO2ₙ in the segments is expressed in relation to an estimated fraction b of CO2 in air in each segment k.

The PCO2 of blood draining the lungs (PtcCO2) is dependent on the gravity-induced perfusion and ventilation gradients, as described by the above functions g and h. For each breath, the PCO2 in each lung segment k (PkCO2,n) is calculated according to Equations 13-18. At FRC, the amount of CO2 in lung segment k (F) is determined by the CO2 content in the lung capillaries, in the FRC and in the VD

F=f(PkCO2,n−1)w(k)Vcap+cPkCO2,n−1w(k)FRC+cPETCO2,n−1w(k)VD

with the weight function w and conversion factor c as described above. The contribution of CO2 in dead space (the right-most term) is computed noting that end-tidal air from the previous breath is returned to the lungs from dead space. The amount of CO2 carried to the lungs from the venous compartment (G) is given by

G=[CO2]v,n−1SVng(k) Equation 14

The ratio a of [CO2] in blood and [CO2] in air is approximated from the previous breath, n−1, according to

a=f(PETCO2,n−1)/(cPETCO2,n−1) Equation 16

The ratio b of the end-tidal amount of CO2 in air and the total amount of CO2 is given by

b=(w(k)FRC+h(k)VTn)/(a(w(k)Vcap+g(k)SVn)+w(k)FRC+h(k)VTn) Equation 16

The end-tidal [CO2] in each lung compartment k is determined by the total amount of CO2 (F+G), which is distributed over air and blood with ratio b, and the end tidal volume of air in compartment k

[CO2]k,n=b(F+G)/(w(k)FRC+h(k)VTn) Equation 17

A simple conversion using the above constant c then yields PkCO2,n. The PETCO2 depends on the distribution of tidal volume, which is given by the fraction h(k),k=1 . . . 9, and differs between the supine and the standing position, and is computed as

$$PETCO 2 , n = {\sum }_{k = 1}^{9} h ( k ) PkCO 2 , n \qquad Equation 18$$

In conjunction with the above described model, through direct calculation, or via iterative testing, or via look up tables the heart stroke volume SVn can be determined in consistency with the model, for each breath.

Application of the Model for the Supine Position

For the supine position, measured CO2 partial pressure in the expiration can be related to the CO2 partial pressure in the lung-compartments. CO2 pressure as a percentage of total air pressure corresponds with CO2 concentration (also as percentage). With respect to CO2 equilibrium curve the CO2 pressure in blood corresponds with a much greater CO2 concentration (percentage), which can be calculated as according to the function of Equation 1 here above. For the supine position, the partial CO2 pressure in the lungs is equal for all segments and also to the measured CO2 partial pressure in the expiratory volume. Hence, the CO2 concentration in blood can be obtained straightforwardly. The blood volume responsible for producing the CO2 can be expressed as the sum of the cardiac output SV plus the capillary volume Vcap. The amount of dissolved CO2 is determined by the CO2 production, which can be estimated. Thus, the cardiac output can be derived by measuring expired CO2 air partial pressure, relating this to a CO2 concentration in the blood using Equation 1, and deriving a cardiac output by dividing the CO2 production by the CO2 concentration in blood, and subtracting an estimated capillary volume of the lungs:

SV=((CO2 production per breath/[CO2]blood)−Vcap) Equation 19

Using a measured respiratory frequency and the heart stroke rate, this value can easily be converted to a cardiac output value per heart stroke. It will be clear to those skilled in the art that the invention is not limited to the exemplary embodiments described with reference to the drawings but may comprise all kinds of variations thereof. Such variations are deemed to fall within the scope of protection of the appended claims.

1. A method of determining the cardiac output of a human heart, comprising:
providing a sensor for measuring a breathing tidal volume;
providing a sensor for measuring a CO2 partial pressure in the breathing tidal volume;
providing a circulation model for relating a measured CO2 partial pressure, an oxygen uptake and a breathing tidal volume to a heart stroke volume per breath; and
providing a processor for inputting a measured breathing tidal volume, an oxygen uptake and a measured CO2 partial pressure in said circulation model, and outputting said heart stroke volume per breath consistent with said circulation model.

2. A method according to claim 1 further comprising:
providing a sensor for measuring said oxygen uptake from the breathing tidal volume.

3. A method according to claim 1, further comprising measuring said CO2 partial pressure as an end tidal CO2 partial pressure in an expiratory tidal volume.

4. A method according to claim 1, further comprising measuring said CO2 partial pressure as a time-integrated CO2 partial pressure in an expiratory tidal volume.

5. A method according to claim 1, wherein said circulation model defines a distribution of apical and basal lung segments, each segment defining a predetermined ventilation perfusion ratio (V/Q), and where the heart stroke volume per breath n SVₙ is calculated to be consistent with an estimated fraction b of CO2 in air in each segment, derived from a measured end tidal CO2 partial pressure and pulmonary oxygen uptake.

6. A method according to claim 1, wherein said circulation model defines a circulated total blood volume and a ventilated total air volume and determines a summed CO2 content in said total blood volume and ventilated total air volume.

7. A method according to claim 2, wherein said segmented circulation model determines:
a segmented perfusion contribution g to CO2 content for a heart stroke volume SV per breath of circulated blood in the lungs;
a segmented ventilation contribution h to CO2 content for an expiratory tidal volume VT; and
a segmented contribution w to CO2 content for a functional residual capacity FRC and a capillary blood volume in the lungs Vcap.

8. A method according to claim 7, wherein said segmented perfusion contribution g is assumed constant in the supine position; and increases substantially linearly from top to basal lung segment in the upright position.

9. A method according to claim 7, wherein said segmented ventilation contribution h is assumed constant in the supine position; and decreases substantially exponentially from top to basal lung segment in the upright position.

10. A method according to claim 7, wherein said segmented contribution w decreases substantially exponentially from top to basal lung segment in the upright position.

11. A method according to claim 2, wherein said circulation model defines a circulated total blood volume by a venous compartment Vv; an arterial compartnent Va; and a fixed blood volume of segmented lung capillaries Vcap; and a variable heart stroke volume per breath SVₙ distributed over the segments; and wherein said ventilated total air volume comprises a fixed volume of a segmented functional residual capacity FRC; a variable expiratory tidal volume VTₙ distributed over the segments; and an anatomical dead space VD.

12. A method according to claim 11, wherein, for each breath n, a CO2 amount in each segment k of said segmented lung model is expressed as the amount F of CO2 in the lung capillaries Vcap, in the functional residual capacity FRC, and in the anatomical dead space VD, as a function of an estimated CO2 partial pressure PₖCO2ₙ in the segments k; plus the amount G of CO2 carried to the lungs from the venous compartment by the heart stroke volume SVₙ; and where the estimated CO2 partial pressure PkCO2/ₙ in the segments is expressed in relation to an estimated fraction b of CO2 in air in each segment k.

13. A method according to claim 12 where said estimated fraction b is expressed by
b=(w(k)×FRC+h(k)×VTn)/(a(w(k)×Vcap+g(k)×SVₙ)+w(k)×FRC+h(k)×VTₙ))
where a defines a ratio of CO2 content in blood and air; the function w(k) is expressed, for k ranging from 1 to 9, increasing from top to basal lung segment, by:
w(k)=0.10055(1.36708−exp(−0.3393k));
h(k) is expressed, for k ranging from 1 to 9, increasing from top to basal lung segment, by:
$$\begin{matrix}h ( k ) = 1 / 9 in the supine position ; and \\ = 0.226 ( 1.102 - exp ( - 0.1063 k ) in the upright position ; and\end{matrix}$$
g(k) is expressed, for k ranging from 1 to 9, increasing from top to basal lung segment, by:
$$\begin{matrix}g ( k ) = 1 / 9 in the supine position ; and \\ = - 0.0205 + 0.0263 k in the upright position .\end{matrix}$$

14. A method according to claim 13, wherein said ratio a is approximated by
a=f(PETCO2ₙ₋₁)/(c×PETCO2ₙ₋₁);
where the function f represents a function relating blood CO2 content ([CO2]) to a measured end tidal blood partial CO2 pressure (PETCO2ₙ₋₁) of a previous breath; and c represents a conversion factor for converting the CO2 partial pressure to a CO2 content in air.

15. A method according to claim 14; wherein the function f is expressed as
f(x)=0.53(1.266−exp(−0.0257x)).

16. A method according to claim 12, where the amounts F and G are expressed for each segment k as:
F=f(PₖCO2ₙ₋₁)×w(k)×Vcap+c×PkCO2ₙ₋₁×w(k)×FRC+c×PETCO2,n−1×w(k)×VD;
G=[CO2]_{v,n-1}×SVₙ×g(k);
where the function w(k) is expressed, for k ranging from 1 to 9, increasing from top to basal lung segment, by:
w(k)=0.10055(1.36708−exp(−0.3393k));
g(k) is expressed, for k ranging from 1 to 9, increasing from top to basal lung segment, by:—
$$\begin{matrix}g ( k ) = 1 / 9 in the supine position ; and \\ = - 0.0205 + 0.0263 k in the upright position ;\end{matrix}$$
where the function f represents a function relating blood CO2 content ([CO2]) to blood partial CO2 pressure (PCO2); PkCO2,n is an estimated CO2 partial pressure in segment k; PETCO2 is the end tidal measured CO2 partial pressure; c represents a conversion factor for converting the CO2 partial pressure to a CO2 content in air; and [CO2]_{v,n} is the CO2 content in the venous compartment.

17. A method according to claim 11, wherein for each breath n, a variation in CO2 in said venous compartment Vv is expressed by the amount A that arrives from the arterial compartment Va plus the amount B of CO2 created by the basal metabolism minus the amount C that exits the venous compartment; where the sum of CO2 created by the basal metabolism is expressed as a function of the oxygen uptake VO2 per breath.

18. A method according to claim 17, wherein said CO2 variation in the venous compartment Vv is expressed by:
[CO2]_{v,n}−[CO2]_{v,n-1}=(A+B−C)/V_{V}
where
A=[C02]_{a,n-1}×SVₙ
B=VO2×RQ
C=[C02]_{v,n-1}×SVₙ
where [C02]_{v,n} is the CO2 content in the venous compartment; [CO2]ₐ is the CO2 content in the arterial compartment; VO2 is the oxygen extraction per breath n and RQ is the respiratory quotient, which is set at 0.9

19. A method according to claim 11, wherein for each breath n, a variation in CO2 in said arterial compartment Va is expressed as the amount of CO2 arriving from the lungs minus the amount that exits the arterial compartment Va; where the amount of CO2 arriving from the lungs is estimated from the end-tidal partial CO2 pressure in each lung segment.

20. A method according to claim 19, wherein said CO2 variation in the arterial compartment Va is expressed by:
[CO2]_{a,n}−[CO2]_{a,n-1}=(D−E)/Va;
where [CO2]_{a,n} is the CO2 content in the arterial compartment for breath n;
$$D = {\sum }_{k} f ( {P}_{k} CO {2}_{n - 1} ) \times g ( k ) \times {SV}_{n} ;$$
where f is a function that relates blood CO2 content to the blood partial CO2 pressure P_{CO2} in a lung segment k and g defines the perfusion contribution of SV over the k lung segments; and
E=[C02]_{a,n-1}X SVₙ.

21. A method according to claim 20; wherein the function f is expressed as
f(x)=0.53(1.266−exp(−0.0257x)); and g is expressed, for k ranging from 1 to 9, increasing from top to basal lung segment, by:
$$\begin{matrix}g ( k ) = 1 / 9 in the supine position ; and \\ = - 0.0205 + 0.0263 k in the upright position .\end{matrix}$$

22. A ventilation unit for actively or passively ventilating the lungs; the unit comprising a breathing mask piece and further comprising:
a respiratory detector for measuring an expiratory tidal volume and respiratory rate;
an oxygen sensor for measuring an oxygen uptake from the expiratory tidal volume;
a CO2 sensor for measuring a CO2 partial pressure in the expiratory tidal volume; and
a processor programmed in consistency with a circulation model, for relating a measured CO2 partial pressure, expiratory tidal volume and oxygen uptake to a heart stroke volume per breath;
wherein the respiratory detector, the oxygen sensor and the C02 sensor are arranged to be coupled to said processor for inputting a measured expiratory tidal volume; a measured oxygen uptake VO2 and a measured CO2 partial pressure, and the processor arranged to output a heart stroke volume per breath to an output unit consistent with said circulation model.

23. A ventilation unit according to claim 22, A method according to claim 1, wherein said circulation model defines a distribution of apical and basal lung segments, each segment defining a predetermined ventilation perfusion ratio (V/Q), and where the heart stroke volume per breath n SVₙ is calculated to be consistent with an estimated fraction 6 of CO2 in air in each segment, derived from a measured end tidal CO2 partial pressure and pulmonary oxygen uptake.

24. A ventilation unit according to claim 22, where said processor is arranged to provide:
an estimated CO2 production based on the measured oxygen uptake;
a CO2 concentration in blood based on the measured CO2 partial pressure; and
a heart stroke volume which is determined by dividing the CO2 production by the CO2 concentration in blood, and subtracting an estimated capillary volume of the lungs.
